SEQUENCE LISTING (12 sequences)

SEQ ID NO: 1 - DNA, 15 bp, Mus musculus
aactatggtgtacac

SEQ ID NO: 2 - protein, 5 aa, Mus musculus
NYGVH

SEQ ID NO: 3 - DNA, 48 bp, Mus musculus
gtgatatggagtggtggaaacacagactataatacacctttcacatcc

SEQ ID NO: 4 - protein, 16 aa, Mus musculus
VIWSGGNTDYNTPFTS

SEQ ID NO: 5 - DNA, 33 bp, Mus musculus
gccctcacctactatgattacgagtttgcttac

SEQ ID NO: 6 - protein, 11 aa, Mus musculus
ALTYYDYEFAY

SEQ ID NO: 7 - DNA, 33 bp, Mus musculus
agggccagtcagagtattggcacaaacatacac

SEQ ID NO: 8 - protein, 11 aa, Mus musculus
RASQSIGTNIH

SEQ ID NO: 9 - DNA, 18 bp, Mus musculus
gcttctgagtctatctct

SEQ ID NO: 10 - protein, 6 aa, Mus musculus
ASESIS

SEQ ID NO: 11 - DNA, 27 bp, Mus musculus
caacaaaataataactggccaaccacg

SEQ ID NO: 12 - protein, 9 aa, Mus musculus
QQNNNWPTT

(19) United States
(12) Patent Application Publication
(10) Pub. No.: US 2009/0297509 A1
(43) Pub. Date: Dec. 3, 2009

(54) Treatment of human tumors with radiation and inhibitors of growth factor receptor tyrosine kinases

(75) Inventors: Harlan W. Waksal, Montclair, NJ (US); Mansoor N. Saleh, Birmingham, AL (US); Francisco Robert, Birmingham, AL (US); Donald Jay Buchsbaum, Birmingham, AL (US)

Correspondence Address: ELI LILLY & COMPANY, PATENT DIVISION, P.O. BOX 6288, INDIANAPOLIS, IN, 46206-6288, US

(73) Assignee: ImClone Systems Incorporated, New York, NY (US)

(21) Appl. No.: 12/321,948

(22) Filed: Jan. 26, 2009

Related U.S. Application Data
(63) Continuation of application No. 10/661,881, filed on Sep. 11, 2003, now abandoned.
Application No. 10/661,881 is a division of application No. 09/312,286, filed on May 14, 1999, now abandoned.
(60) Provisional application No. 60/085,613, filed on May 15, 1998.

(51) Int. Cl. A61K 39/395; A61P 35/00

(52) U.S. Cl. 424/133.1; 424/141.1

(57) ABSTRACT

A method to inhibit the growth of tumors in human patients, comprising treating the human patients with an effective amount of a combination of radiation and a non-radiolabeled protein receptor tyrosine kinase inhibitor, the overexpression of which can lead to tumorigenesis.

This application is a continuation of U.S. application Ser. No. 10/661,881, filed on Sep. 11, 2003, which is divisional of U.S. application Ser. No. 09/312,286 filed on May 14, 1999, which claims the benefit of U.S. Provisional Application No. 60/085,613 filed May 15, 1998, all of which are incorporated herein by reference.

Normal cells proliferate by the highly controlled activation of growth factor receptors by their respective ligands. An example of such receptors are the growth factor receptor tyrosine kinases.

Cancer cells also proliferate by the activation of growth factor receptors, but lose the careful control of normal proliferation. The loss of control may be caused by numerous factors, such as the autocrine secretion of growth factors, increased expression of receptors, and autonomous activation of biochemical pathways regulated by growth factors.

Some examples of receptors involved in tumorigenesis are the receptors for epidermal growth factor (EGFR), platelet-derived growth factor (PDGFR), insulin like growth factor (IGFR), nerve growth factor (NGFR), and fibroblast growth factor (FGF).

Members of the epidermal growth factor (EGF) receptor family are particularly important growth factor receptor tyrosine kinases associated with tumorigenesis of epidermal cells. The first member of the EGF receptor family to be discovered was the glycoprotein having an apparent molecular weight of approximately 165 kD. This glycoprotein, which was described by Mendelsohn et al. in U.S. Pat. No. 4,943,533, is known as the EGF receptor (EGFR) and also as human EGF receptor-1 (HER1).

The EGFR is overexpressed on many types of epidermoid tumor cells. EGF and transforming growth factor alpha (TGF-alpha) are two known ligands of EGFR. Examples of tumors that express EGF receptors include glioblastomas, as well as cancers of the lung, breast, head and neck, and bladder. The amplification and/or overexpression of the EGF receptors on the membranes of tumor cells is associated with a poor prognosis.

Some progress has been made in treating cancer. Useful treatments include those that rely on the programmed death of cells that have suffered DNA damage. The programmed death of cells is known as apoptosis.

Treatments of cancer traditionally include chemotherapy or radiation therapy. Some examples of chemotherapeutic agents include doxorubicin, cis-platin, and taxol. The radiation can be either from an external beam or from a source placed inside a patient, i.e., brachytherapy.

Another type of treatment includes inhibitors of growth factors or growth factor receptors involved in the proliferation of cells. Such inhibitors neutralize the activity of the growth factor or receptor, and inhibit the growth of tumors that express the receptor.

For example, U.S. Pat. No. 4,943,533 describes a murine monoclonal antibody called 225 that binds to the EGF receptor. The patent is assigned to the University of California and licensed exclusively to ImClone Systems Incorporated. The 225 antibody is able to inhibit the growth of cultured EGFR-expressing tumor lines as well as the growth of these tumors in vivo when grown as xenografts in nude mice. See Masui et al., Cancer Res. 44, 5592-5598 (1986).

Similarly, Prewett et al. reported the inhibition of tumor progression of well-established prostate tumor xenografts in mice with a chimeric form of the anti-EGFR 225 monoclonal antibody discussed above. The chimeric form is called c225. Journal of Immunotherapy 19, 419-427 (1997).

A disadvantage of using murine monoclonal antibodies in human therapy is the possibility of a human anti-mouse antibody (HAMA) response due to the presence of mouse Ig sequences. This disadvantage can be minimized by replacing the entire constant region of a murine (or other non-human mammalian) antibody with that of a human constant region. Replacement of the constant regions of a murine antibody with human sequences is usually referred to as chimerization.

The chimerization process can be made even more effective by also replacing the framework variable regions of a murine antibody with the corresponding human sequences. The framework variable regions are the variable regions of an antibody other than the hypervariable regions. The hypervariable regions are also known as the complementarity-determining regions (CDRs).

The replacement of the constant regions and framework variable regions with human sequences is usually referred to as humanization. The humanized antibody is less immunogenic (i.e. elicits less of a HAMA response) as more murine sequences are replaced by human sequences. Unfortunately, both the cost and effort increase as more regions of a murine antibodies are replaced by human sequences.

Another approach to reducing the immunogenicity of antibodies is the use of antibody fragments. For example, an article by Aboud-Pirak et al., Journal of the National Cancer Institute 80, 1605-1611 (1988), compares the anti-tumor effect of an anti-EGF receptor antibody called 108.4 with fragments of the antibody. The tumor model was based on KB cells as xenografts in nude mice. KB cells are derived from human oral epidermoid carcinomas, and express elevated levels of EGF receptors.

Aboud-Pirak et al. found that both the antibody and the bivalent F(ab′)₂ fragment retarded tumor growth in vivo, although the F(ab′)₂ fragment was less efficient. The monovalent Fab fragment of the antibody, whose ability to bind the cell-associated receptor was conserved, did not, however, retard tumor growth.

Attempts have also been made to improve cancer treatments by combining some of the techniques mentioned above. For example, Baselga et al. reported anti-tumor effects of the chemotherapeutic agent doxorubicin with anti-EGFR monoclonal antibodies in the Journal of the National Cancer Institute 85, 1327-1333 (1993).

Others have attempted to enhance the sensitivity of cancer cells to radiation by combining the radiation with adjuvants. For example, Bonnen, U.S. Pat. No. 4,846,782, reported increased sensitivity of human cancers to radiation when the radiation was combined with interferon. Snelling et al. reported a minor improvement in the radiation treatment of patients with astrocytomas with anaplastic foci when the radiation was combined with an anti-EGFR monoclonal antibody radiolabeled with iodine-1125 in a phase II clinical trial. See Hybridoma 14, 111-114 (1995).

Similarly, Balaban et al. reported the ability of anti-EGFR monoclonal antibodies to sensitize human squamous carcinoma xenografts in mice to radiation when the radiation treatment was preceded by administration of an anti-EGFR antibody called LA22. See Biochimica et Biophysica Acta 1314, 147-156 (1996). Saleh et al. also reported better tumor control in vitro and in mice when radiation therapy was augmented with anti-EGFR monoclonal antibodies. Saleh et al. concluded that: “Further studies . . . may lead to a novel combined modality RT/Mab therapy.” See abstract 4197 in the proceedings of the American Association for Cancer Research 37, 612 (1996).

While some of the studies described above suggest further experiments in humans, the results reported are for models in mice. Such models do not necessarily provide a reasonable expectation for success in humans. As was stated in the New York Times of May 3, 1998, in regard to the spectacular success reported by Judah Folkman in treating tumors in mice with angiostatin and endostatin: “Until patients take them, he said, it is dangerous to make predictions. All he knows, Dr. Folkman said, is that ‘if you have cancer and you are a mouse, we can take good care of you.’” See page 1 of the New York Times of May 3, 1998.

Cancer continues to be a major health problem. The objective of the present invention is to provide an improved method for treating certain cancers in humans.

### SUMMARY OF THE INVENTION

This, and other objectives as will be apparent to those having ordinary skill in the art, have been achieved by providing a new method to inhibit the growth of tumors in human patients. The method comprises treating the human patients with an effective amount of a combination of radiation and a non-radiolabeled protein receptor tyrosine kinase inhibitor, the overexpression of which can lead to tumorigenesis.

### DETAILED DESCRIPTION OF THE INVENTION

The present invention provides an improved method for treating tumors, particularly malignant tumors, in human patients who have cancer, or are at risk of developing cancer. The types of tumors that can be treated in accordance with the invention are tumors that overexpress one or more growth factor receptor tyrosine kinases. Some examples of growth factor receptor tyrosine kinases that can lead to tumorigenesis if overexpressed include the EGFR family of receptors, PDGFR family of receptors, IGFR family of receptors, NGFR family of receptors, TGF family of receptors, and FGFR family of receptors.

The EGFR family of receptors includes EGFR, which is also referred to in the literature as HER1; HER2, which is also referred to in the literature as Neu, c-erbB-2, and p185erbB-2; erbB-3 and erbB-4. In this specification, EGFR refers to the EGFR family of receptors. The specific member of the EGFR family of receptors that is also called EGFR will be referred to as EGFR/HER1.

The PDGFR family of receptors includes PDGFRα and PDGFRβ. The IGF family of receptors includes IGFR-1. Members of the FGFR family include FGFR-1, FGFR-2, FGFR-3, and FGFR-4. The TGFR family of receptors includes TGFRα and TGFRβ.

Any type of tumor that overexpresses at least one growth factor receptor tyrosine kinase, the overexpression of which can lead to tumorigenesis, can be treated in accordance with the method of the invention. These types of tumor include carcinomas, gliomas, sarcomas, adenocarcinomas, adenosarcomas and adenomas.

Such tumors occur in virtually all parts of the human body, including every organ. The tumors may, for example, be present in the breast, lung, colon, kidney, bladder, head and neck, ovary, prostate, brain, pancreas, skin, bone, bone marrow, blood, thymus, uterus, testicles, cervix, and liver. For example, tumors that overexpress the EGF receptor include breast, lung, colon, kidney, bladder, head and neck, especially squamous cell carcinoma of the head and neck, ovary, prostate, and brain.

The tumors are treated with a combination of radiation therapy and a non-radiolabeled growth factor receptor tyrosine kinase inhibitor. For the purposes of this specification, the inhibition of a growth factor receptor tyrosine kinase means that the growth of cells overexpressing such receptors is inhibited.

No particular mechanism of inhibition is implied. Nevertheless, growth factor receptor tyrosine kinases are generally activated by means of phosphorylation events. Accordingly, phosphorylation assays are useful in predicting the inhibitors useful in the present invention. Some useful assays for tyrosine kinase activity are described in Panek et al., Journal of Pharmacology and Experimental Therapeutics 283, 1433-1444 (1997) and in Batley et al., Life Sciences 62, 143-150 (1998). The description of these assays is incorporated herein by reference.

In the preferred embodiment, there is synergy when tumors in human patients are treated with a combination of an inhibitor of a growth factor receptor tyrosine kinase and radiation, as described herein. In other words, the inhibition of tumor growth from the combined treatment with an inhibitor and radiation is better than would be expected from treatment with either the inhibitor or radiation alone. Synergy may be shown, for example, by greater inhibition of tumor growth with the combined treatment than would be expected from treatment with either inhibitor or radiation alone. Preferably, synergy is demonstrated by remission of the cancer with the combined treatment with inhibitor and radiation where remission is not expected from treatment with either inhibitor or radiation alone.

The source of radiation can be either external or internal to the patient being treated. When the source is external to the patient, the therapy is known as external beam radiation therapy (EBRT). When the source of radiation is internal to the patient, the treatment is called brachytherapy (BT).

The radiation is administered in accordance with well known standard techniques with standard equipment manufactured for this purpose, such as AECL Theratron and Varian Clinac. The dose of radiation depends on numerous factors as is well known in the art. Such factors include the organ being treated, the healthy organs in the path of the radiation that might inadvertently be adversely affected, the tolerance of the patient for radiation therapy, and the area of the body in need of treatment. The dose will typically be between 1 and 100 Gy, and more particularly between 2 and 80 Gy. Some doses that have been reported include 35 Gy to the spinal cord, 15 Gy to the kidneys, 20 Gy to the liver, and 65-80 Gy to the prostate. It should be emphasized, however, that the invention is not limited to any particular dose. The dose will be determined by the treating physician in accordance with the particular factors in a given situation, including the factors mentioned above.

The distance between the source of the external radiation and the point of entry into the patient may be any distance that represents an acceptable balance between killing target cells and minimizing side effects. Typically, the source of the external radiation is between 70 and 100 cm from the point of entry into the patient.

Brachytherapy is generally carried out by placing the source of radiation in the patient. Typically, the source of radiation is placed approximately 0-3 cm from the tissue being treated. Known techniques include interstitial, intercavitary, and surface brachytherapy. The radioactive seeds can be implanted permanently or temporarily. Some typical radioactive atoms that have been used in permanent implants include iodine-125 and radon. Some typical radioactive atoms that have been used in temporary implants include radium, cesium-137, and iridium-192. Some additional radioactive atoms that have been used in brachytherapy include americium-241 and gold-198.

The dose of radiation for brachytherapy can be the same as that mentioned above for external beam radiation therapy. In addition to the factors mentioned above for determining the dose of external beam radiation therapy, the nature of the radioactive atom used is also taken into account in determining the dose of brachytherapy.

The growth factor receptor tyrosine kinase inhibitor is administered before, during, or after commencing the radiation therapy, as well as any combination thereof, i.e. before and during, before and after, during and after, or before, during, and after commencing the radiation therapy. The antibody is typically administered between 1 and 30 days, preferably between 3 and 20 days, more preferably between 5 and 12 days before commencing radiation therapy and/or termination of external beam radiation therapy.

Any non-radiolabeled inhibitor of a growth factor receptor tyrosine kinase, the overexpression of which can be tumorigenic, is useful in the method of the invention. The types of tumors that overexpress such receptors have been discussed above. The inhibitors may be biological molecules or small molecules.

Biological inhibitors include proteins or nucleic acid molecules that inhibit the growth of cells that overexpress a growth factor receptor tyrosine kinase. Most typically, biological molecules are antibodies, or functional equivalents of antibodies.

Functional equivalents of antibodies have binding characteristics comparable to those of antibodies, and inhibit the growth of cells that overexpress growth factor receptor tyrosine kinase receptors. Such functional equivalents include, for example, chimerized, humanized and single chain antibodies as well as fragments thereof.

Functional equivalents of antibodies include polypeptides with amino acid sequences substantially the same as the amino acid sequence of the variable or hypervariable regions of the antibodies of the invention. “Substantially the same” amino acid sequence is defined herein as a sequence with at least 70%, preferably at least about 80%, and more preferably at least about 90% homology to another amino acid sequence, as determined by the FASTA search method in accordance with Pearson and Lipman, Proc. Natl. Acad. Sci. USA 85, 2444-2448 (1988). The DNA molecules that encode functional equivalents of antibodies typically bind under stringent conditions to the DNA of the antibodies.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody. The variable region other than the hypervariable region, e.g. the framework variable region, and the constant region of a humanized antibody are those of a human antibody.

For the purposes of this application, suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates. Mice are preferred.

Functional equivalents further include fragments of antibodies that have binding characteristics that are the same as, or are comparable to, those of the whole antibody. Suitable fragments of the antibody include any fragment that comprises a sufficient portion of the hypervariable (i.e. complementarity determining) region to bind specifically, and with sufficient affinity, to a growth factor receptor tyrosine kinase to inhibit growth of cells that overexpress such receptors.

Such fragments may, for example, contain one or both Fab fragments or the F(ab′)₂ fragment. Preferably the antibody fragments contain all six complementarity determining regions of the whole antibody, although functional fragments containing fewer than all of such regions, such as three, four or five CDRs, are also included.

The preferred fragments are single chain antibodies, or Fv fragments. Single chain antibodies are polypeptides that comprise at least the variable region of the heavy chain of the antibody linked to the variable region of the light chain, with or without an interconnecting linker. Thus, Fv fragment comprises the entire antibody combining site. These chains may be produced in bacteria or in eucaryotic cells.

The antibodies and functional equivalents may be members of any class of immunoglobulins, such as: IgG, IgM, IgA, IgD, or IgE, and the subclasses thereof. The preferred antibodies are members of the IgG1 subclass. The functional equivalents may also be equivalents of combinations of any of the above classes and subclasses.

Antibodies may be made from the desired receptor by methods that are well known in the art. The receptors are either commercially available, or can be isolated by well known methods. For example, methods for isolating and purifying EGFR are found in Spada, U.S. Pat. No. 5,646,153 starting at column 41, line 55. Methods for isolating and purifying FGFR are found in Williams et al., U.S. Pat. No. 5,707,632 in examples 3 and 4. The methods for isolating and purifying EGFR and FGFR described in the Spada and Williams et al. patents are incorporated herein by reference.

Methods for making monoclonal antibodies include the immunological method described by Kohler and Milstein in Nature 256, 495-497 (1975) and by Campbell in “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985). The recombinant DNA method described by Huse et al. in Science 246, 1275-1281 (1989) is also suitable.

Briefly, in order to produce monoclonal antibodies, a host mammal is inoculated with a receptor or a fragment of a receptor, as described above, and then, optionally, boosted. In order to be useful, the receptor fragment must contain sufficient amino acid residues to define the epitope of the molecule being detected. If the fragment is too short to be immunogenic, it may be conjugated to a carrier molecule. Some suitable carrier molecules include keyhold limpet hemocyanin and bovine serum albumen. Conjugation may be carried out by methods known in the art. One such method is to combine a cysteine residue of the fragment with a cysteine residue on the carrier molecule.

Spleens are collected from the inoculated mammals a few days after the final boost. Cell suspensions from the spleens are fused with a tumor cell. The resulting hybridoma cells that express the antibodies are isolated, grown, and maintained in culture.

Suitable monoclonal antibodies as well as growth factor receptor tyrosine kinases for making them are also available from commercial sources, for example, from Upstate Biotechnology, Santa Cruz Biotechnology of Santa Cruz, Calif., Transduction Laboratories of Lexington, Ky., R&D Systems Inc of Minneapolis, Minn., and Dako Corporation of Carpinteria, Calif.

Methods for making chimeric and humanized antibodies are also known in the art. For example, methods for making chimeric antibodies include those described in U.S. patents by Boss (Celltech) and by Cabilly (Genentech). See U.S. Pat. Nos. 4,816,397 and 4,816,567, respectively. Methods for making humanized antibodies are described, for example, in Winter, U.S. Pat. No. 5,225,539.

The preferred method for the humanization of antibodies is called CDR-grafting. In CDR-grafting, the regions of the mouse antibody that are directly involved in binding to antigen, the complementarity determining region or CDRs, are grafted into human variable regions to create “reshaped human” variable regions. These fully humanized variable regions are then joined to human constant regions to create complete “fully humanized” antibodies.

In order to create fully humanized antibodies that bind well to antigen, it is advantageous to design the reshaped human variable regions carefully. The human variable regions into which the CDRs will be grafted should be carefully selected, and it is usually necessary to make a few amino acid changes at critical positions within the framework regions (FRs) of the human variable regions.

For example, the reshaped human variable regions may include up to ten, amino acid changes in the FRs of the selected human light chain variable region, and as many as twelve amino acid changes in the FRs of the selected human heavy chain variable region. The DNA sequences coding for these reshaped human heavy and light chain variable region genes are joined to DNA sequences coding for the human heavy and light chain constant region genes, preferably γ1 and κ, respectively. The reshaped humanized antibody is then expressed in mammalian cells and its affinity for its target compared with that of the corresponding murine antibody and chimeric antibody.

Methods for selecting the residues of the humanized antibody to be substituted and for making the substitutions are well known in the art. See, for example, Co et al., Nature 351, 501-502 (1992); Queen et al., Proc. Natl. Acad. Sci. 86, 10029-1003 (1989) and Rodrigues et al., Int. J. Cancer, Supplement 7, 45-50 (1992). A method for humanizing and reshaping the 225 anti-EGFR monoclonal antibody described by Goldstein et al. in PCT application WO 96/40210. This method can be adapted to humanizing and reshaping antibodies against other growth factor receptor tyrosine kinases.

Methods for making single chain antibodies are also known in the art. Some suitable examples include those described by Wels et al. in European patent application 502 812 and Int. J. Cancer 60, 137-144 (1995).

Other methods for producing the functional equivalents described above are disclosed in PCT Application WO 93/21319, European Patent Application 239 400, PCT Application WO 89/09622, European Patent Application 338 745, U.S. Pat. No. 5,658,570, U.S. Pat. No. 5,693,780, and European Patent Application EP 332 424.

Preferred antibodies are those that inhibit the EGF receptor. Preferred EGFR antibodies are the chimerized, humanized, and single chain antibodies derived from a murine antibody called 225, which is described in U.S. Pat. No. 4,943,533. The patent is assigned to the University of California and licensed exclusively to ImClone Systems Incorporated.

The 225 antibody is able to inhibit the growth of cultured EGFR/HER1-expressing tumor cells in vitro as well as in vivo when grown as xenografts in nude mice. See Masui et al., Cancer Res. 44, 5592-5598 (1986). More recently, a treatment regimen combining 225 plus doxorubicin or cis-platin exhibited therapeutic synergy against several well established human xenograft models in mice. Basalga et al., J. Natl. Cancer Inst. 85, 1327-1333 (1993).

The chimerized, humanized, and single chain antibodies derived from murine antibody 225 can be made from the 225 antibody, which is available from the ATCC. Alternatively, the various fragments needed to prepare the chimerized, humanized, and single chain 225 antibodies can be synthesized from the sequence provided in Wels et al. in Int. J. Cancer 60, 137-144 (1995). Chimerized 225 antibody (c225) can be made in accordance with the methods described above. Humanized 225 antibody can be prepared in accordance with the method described in example IV of PCT application WO 96/40210, which is incorporated herein by reference. Single chain 225 antibodies (Fv225) can be made in accordance with methods described by Wels et al. in Int. J. Cancer 60, 137-144 (1995) and in European patent application 502 812.

The sequences of the hypervariable (CDR) regions of the light and heavy chain are reproduced below. The amino acid sequence is indicated below the nucleotide sequence.

HEAVY CHAIN HYPERVARIABLE REGIONS (VH):
CDR1

(SEQ ID 1)

AACTATGGTGTACAC

(SEQ ID 2)

N Y G V H
CDR2

(SEQ ID 3)

GTGATATGGAGTGGTGGAAACACAGACTATAATACACCTTTCACATCC

(SEQ ID 4)

V I W S G G N T D Y N T P F T S
CDR3

(SEQ ID 5)

GCCCTCACCTACTATGATTACGAGTTTGCTTAC

(SEQ ID 6)

A L T Y Y D Y E F A Y
LIGHT CHAIN HYPERVARIABLE REGIONS (VL):
CDR1

(SEQ ID 7)

AGGGCCAGTCAGAGTATTGGCACAAACATACAC

(SEQ ID 8)

R A S Q S I G T N I H
CDR2

(SEQ ID 9)

GCTTCTGAGTCTATCTCT

(SEQ ID 10)

A S E S I S
CDR3

(SEQ ID 11)

CAACAAAATAATAACTGGCCAACCACG

(SEQ ID 12)

Q Q N N N W P T T

In addition to the biological molecules discussed above, the inhibitors useful in the present invention may also be small molecules. For the purposes of this specification, small molecules include any organic or inorganic molecule, other than a biological molecule, that inhibits the growth of cells that overexpress at least one growth factor receptor tyrosine kinase. The small molecules typically have molecular weights less than 500, more typically less than 450. Most of the small molecules are organic molecules that usually comprise carbon, hydrogen and, optionally, oxygen, nitrogen, and/or sulfur atoms.

Numerous small molecules have been described as being useful to inhibit EGFR. For example, Spada et al., U.S. Pat. No. 5,656,655, discloses styryl substituted heteroaryl compounds that inhibit EGFR. The heteroaryl group is a monocyclic ring with one or two heteroatoms, or a bicyclic ring with 1 to about 4 heteroatoms, the compound being optionally substituted or polysubstituted. The compounds disclosed in U.S. Pat. No. 5,656,655 are incorporated herein by reference.

Spada et al., U.S. Pat. No. 5,646,153 discloses bis mono and/or bicyclic aryl heteroaryl carbocyclic and heterocarbocyclic compounds that inhibit EGFR and/or PDGFR. The compounds disclosed in U.S. Pat. No. 5,646,153 are incorporated herein by reference.

Bridges et al., U.S. Pat. No. 5,679,683 discloses tricyclic pyrimidine compounds that inhibit the EGFR. The compounds are fused heterocyclic pyrimidine derivatives described at column 3, line 35 to column 5, line 6. The description of these compounds at column 3, line 35 to column 5, line 6 is incorporated herein by reference.

Barker, U.S. Pat. No. 5,616,582 discloses quinazoline derivatives that have receptor tyrosine kinase inhibitory activity. The compounds disclosed in U.S. Pat. No. 5,616,582 are incorporated herein by reference.

Fry et al., Science 265, 1093-1095 (1994) discloses a compound having a structure that inhibits EGFR. The structure is shown in FIG. 1. The compound shown in FIG. 1 of the Fry et al. article is incorporated herein by reference.

Osherov et al., disclose tyrphostins that inhibit EGFR/HER1 and HER2. The compounds disclosed in the Osherov et al. article, and, in particular, those in Tables I, II, III, and IV are incorporated herein by reference.

Levitzki et al. U.S. Pat. No. 5,196,446, discloses heteroarylethenediyl or heteroarylethenediylaryl compounds that inhibit EGFR. The compounds disclosed in U.S. Pat. No. 5,196,446 from column 2, line 42 to column 3, line 40 are incorporated herein by reference.

Batley et al., Life Sciences 62, 143-150 (1998), disclose a compound called PD161570 that inhibits members of the FGF are family of receptors. PD161570 is identified as t-butyl-3-(6-(2,6-dichlorophenyl)-2-(4-diethylamino-butylamino)-pyrido(2,3-d)pyrimidin-7-yl)urea having the structure shown in FIG. 1 on page 146. The compound described in FIG. 1 on page 146 of the Batley et al. article in Life Sciences 62, 143-150 (1998) is incorporated herein by reference.

Panek, et al., Journal of Pharmacology and Experimental Therapeutics 283, 1433-1444 (1997) disclose a compound identified as PD166285 that inhibits the EGFR, PDGFR, and FGFR families of receptors. PD166285 is identified as 6-(2,6-dichlorophenyl)-2-(4-(2-diethylaminoethoxy)phenylamino)-8-methyl-8H-pyrido(2,3-d)pyrimidin-7-one having the structure shown in FIG. 1 on page 1436. The compound described in FIG. 1 on page 1436 of the Panek et al. article is incorporated herein by reference.

Parrizas, et al., Endocrinology 138, 1427-1433 disclose tyrphostins that inhibit the IGF-1 receptor. The compounds disclosed in the Parrizas et al. article, in particular those in Table 1 on page 1428, are incorporated herein by reference.

The administration of small molecule and biological drugs to human patients is accomplished by methods known in the art. For small molecules, such methods are described in Spada, U.S. Pat. No. 5,646,153 at column 57, line 47 to column 59, line 67. This description of administering small molecules is incorporated herein by reference.

The biological molecules, preferably antibodies and functional equivalents of antibodies, significantly inhibit the growth of tumor cells when administered to a human patient in an effective amount in combination with radiation, as described above. The optimal dose of the antibodies and functional equivalents of antibodies can be determined by physicians based on a number of parameters including, for example, age, sex, weight, severity of the condition being treated, the antibody being administered, and the route of administration. In general, a serum concentration of polypeptides and antibodies that permits saturation of the target receptor is desirable. For example, a concentration in excess of approximately 0.1 nM is normally sufficient. For example, a dose of 100 mg/m² of C225 provides a serum concentration of approximately 20 nM for approximately eight days.

As a rough guideline, doses of antibodies may be given weekly in amounts of 10-300 mg/m². Equivalent doses of antibody fragments should be used at more frequent intervals in order to maintain a serum level in excess of the concentration that permits saturation of the receptors.

Some suitable routes of administration include intravenous, subcutaneous, and intramuscle administration. Intravenous administration is preferred.

The peptides and antibodies of the invention may be administered along with additional pharmaceutically acceptable ingredients. Such ingredients include, for example, adjuvants, such as BCG, immune system stimulators and chemotherapeutic agents, such as those mentioned above.

### Example 1

### Clinical Trial

In a clinical trial, human patients were treated with anti-EGFR chimeric monoclonal antibody c225 at the indicated doses along with 2 Gy (per fraction) of external beam radiation per day, five days a week, for seven weeks, a total of 70 Gy. The results are shown in the table, wherein CR means complete response, PR means partial response, and TBD means to be determined.

TABLE
Clinical Response

| | Dose Level | Clinical | Overall |
|---|---|---|---|
| Patient | (mg/m²) | (Physical Exam) | Response* |
| 1 | 100 | CR | PR |
| 2 | 100 | CR | CR |
| 3 | 100 | CR | CR |
| 4 | 200 | CR | CR |
| 5 | 200 | CR | CR |
| 6 | 200 | CR | PR |
| 7 | 400/200 | PR | CR |
| 8 | 400/200 | CR | CR |
| 9 | 400/200 | CR | PR |
| 10 | 500/250 | CR | PR |
| 11 | 500/250 | CR | PR |
| 12 | 500/250 | CR | TBD |

*Radiographic follow-up ongoing

### Supplemental Enablement

The invention as claimed is enabled in accordance with the above specification and readily available references and starting materials. Nevertheless, Applicants have, on May 13, 1998, re-deposited with the American Type Culture Collection, 12301 Parklawn Drive, Rockville, Md., 20852 USA (ATCC) the hybridoma cell line that produces the murine monoclonal antibody called m225. This antibody was originally deposited in support of U.S. Pat. No. 4,943,533 of Mendelsohn et al. with accession number HB8508.

The re-deposit was made under the provisions of the Budapest Treaty on the International Recognition of the Deposit of Microorganisms for the Purposes of Patent Procedure and the regulations thereunder (Budapest Treaty). This assures maintenance of a viable culture for thirty (30) years from date of deposit. The organism will be made available by ATCC under the terms of the Budapest Treaty, and subject to an agreement between Applicants and ATCC which assures unrestricted availability upon issuance of the pertinent U.S. patent. Availability of the deposited strains is not to be construed as a license to practice the invention in contravention of the rights granted under the authority of any government in accordance with its patent laws.

What is claimed is:

1. A method to inhibit the growth of tumors in human patients, comprising treating the human patients with an effective amount of a combination of radiation and a non-radiolabeled protein receptor tyrosine kinase inhibitor, the overexpression of which can lead to tumorigenesis.

2. A method according to claim 1 wherein the inhibitor is a monoclonal antibody or a fragment that comprises the hypervariable region thereof.

3. A method according to claim 2 wherein the monoclonal antibody is chimerized or humanized.

4. A method according to claim 1 wherein the inhibitor is a small molecule.

5. A method according to claim 1 wherein the protein receptor tyrosine kinase is EGFR, PDGFR, TGF, IGFR, NGFR, or FGFR.

6. A method according to claim 5 wherein the growth factor receptor tyrosine kinase is a member of the EGFR family.

7. A method according to claim 6 wherein the member of the EGFR family is EGFR/HER-1.

8. A method according to claim 6 wherein the member of the EGFR family is HER2.

9. A method according to claim 6 wherein the member of the EGFR family is erbB3.

10. A method according to claim 6 wherein the member of the EGFR family is erbB4.

11. A method according to claim 5 wherein the growth factor receptor tyrosine kinase is a member of the PDGFR family.

12. A method according to claim 11 wherein the member of the PDGFR family is PDGFRα.

13. A method according to claim 11 wherein the member of the PDGFR family is PDGFRβ.

14. A method according to claim 5 wherein the growth factor receptor tyrosine kinase is a member of the FGFR family.

15. A method according to claim 14 wherein the member of the FGFR family is FGFR-1.

16. A method according to claim 14 wherein the member of the FGFR family is FGFR-2.

17. A method according to claim 14 wherein the member of the FGFR family is FGFR-3.

18. A method according to claim 14 wherein the member of the FGFR family is FGFR-4.

19. A method according to claim 5 wherein the growth factor receptor tyrosine kinase is a member of the IGFR family.

20. A method according to claim 19 wherein the member of the IGFR family is IGFR-1.

21. A method according to claim 5 wherein the growth factor receptor tyrosine kinase is a member of the TGF family.

22. A method according to claim 5 wherein the growth factor receptor tyrosine kinase is NGFR.

23. A method according to claim 2 wherein the monoclonal antibody is specific for EGFR/HER1.

24. A method according to claim 23 wherein the monoclonal antibody inhibits EGFR/HER1 phosphorylation.

25. A method according to claim 3 wherein the antibody is specific for EGFR/HER1.

26. A method according to claim 25 wherein the antibody inhibits EGFR/HER1 phosphorylation.

27. A method according to claim 4 wherein the small molecule is specific for EGFR.

28. A method according to claim 27 wherein the small molecule inhibits EGFR phosphorylation.

29. A method according to claim 2 wherein the tumors overexpress EGFR/HER1.

30. A method according to claim 29 wherein the tumors are tumors of the breast, lung, colon, kidney, bladder, head and neck, ovary, prostate, and brain.

31. A method according to claim 2 wherein the antibodies are administered before radiation.

32. A method according to claim 2 wherein the antibodies are administered during radiation.

33. A method according to claim 2 wherein the antibodies are administered after the radiation.

34. A method according to claim 2 wherein the antibodies are administered before and during radiation.

35. A method according to claim 2 wherein the antibodies are administered during and after radiation.

36. A method according to claim 2 wherein the antibodies are administered before and after radiation.

37. A method according to claim 2 wherein the antibodies are administered before, during, and after radiation.

38. A method according to claim 2 wherein the source of the radiation is external to the human patient.

39. A method according to claim 2 wherein the source of radiation is internal to the human patient.
